(19) United States
(12) Patent Application Publication
(10) Pub. No.: US 2014/0363528 A1
(43) Pub. Date: Dec. 11, 2014

(54) COMPOSITION FOR PREVENTING OR AMELIORATING SNORING

(71) Applicant: Pharmacure Health Care AB, Goteborg (SE)

(72) Inventor: Ake Akerman, Brussels (BE)

(21) Appl. No.: 13/912,041

(22) Filed: Jun. 6, 2013

(51) Int. Cl. A61K 9/00; A61K 36/63; A61K 31/355; A61K 36/534; A61K 31/4415; A61K 31/375; A23L 1/30; A61K 36/28

(52) U.S. Cl. CPC A61K 9/0053; A23L 1/3006; A61K 36/63; A61K 36/28; A61K 36/534; A61K 31/4415; A61K 31/375; A61K 31/355
USPC 424/747

(57) ABSTRACT

A composition for preventing or ameliorating snoring includes olive oil, sunflower oil and mentha oil. An agent or food is provided including the composition. A process for the preparation of the composition includes mixing olive oil, sunflower oil and mentha oil, optionally with any other components of the composition, in any order. A method of preventing or ameliorating snoring, includes administering to a patient the composition.

### TECHNICAL FIELD

The present invention relates to a composition for preventing or ameliorating snoring, which can be applied to the pharyngeal region in a simple and easy manner without causing uneasiness or discomfort and further can be taken or used continuously.

### BACKGROUND ART

“Snoring” refers to a sound made when breathing is carried out in the state where the soft palate or the root of the tongue blocks and narrows the upper airway due to a variety of diseases or organic dysfunctions/disorders as well as temporary fatigue, alcohol drinking or the like, and generated by vibration of the blocking obstacle as air passes in and out, and it also refers to a pathological condition in which a condition causing such a sound continues on consecutive days.

Examples of reasons for the narrowing of the upper airway include obesity; large uvula; swollen tonsils or adenoids due to inflammation or the like; large tongue or large soft palate; diseases accompanied by nasal obstruction such as allergic rhinitis, nasal polyp and empyema; and muscle weakness due to aging, fatigue, alcohol drinking or influence of drugs such as sleeping pills.

Snoring considerably affects not only health issues but also social and interpersonal issues. As to the health issues, even when the person is not suffering from diseases such as sleep apnea syndrome or sleep hypopnea pyndrome, snoring may interfere with activity during the day due to degradation of the quality of sleeping. As to the social and interpersonal issues, snoring may cause the person to stay away from group tours due to a sense of shame or interrupt the sleep of his/her roommate.

Among medical devices for preventing or ameliorating snoring, a clip to be inserted into the nostrils to pinch the nasal septum and a mouthpiece are commercially sold. CPAP (Continuous Positive Airway Pressure), which is a treatment device for sleep apnea syndrome, is also believed to have effects of eliminating snoring.

In order to ameliorate nasal obstruction, there are used nasal drops containing a vasoconstrictor such as naphazoline nitrate and tetrahydrozoline hydrochloride. Moreover, for the purpose of moisturizing the nasal mucosa, nasal drops containing glycerol have been used, and a composition containing one or more of eucalyptus oil, mentha oil, peppermint oil, menthol and cineole, as well as the use of suan zao ren tang and the like, have been suggested (Patent Documents 1 and 2).

However, the nasal clip and the mouthpiece may cause uneasiness or discomfort in use, and treatment devices such as CPAP are hardly able to be used in simple and easy manner. As to the nasal drops containing a vasoconstrictor, there are reported side effects such as hypersensitivity, nervousness, elevated blood pressure, stimulation pain, feeling of dryness, nausea and palpitation; therefore, when used continuously for a long term, the nasal drops may cause the nasal mucosa to be thickened. As to the nasal drops for the purpose of moisturizing to the nasal mucosa and the like, when a humectant is used in concentrations so as to give sufficient moisture effect on the nasal mucosa, the nasal drops may stimulate the nasal mucosa, causing inflammation. Furthermore, although snoring continues chronically in most cases, pharmaceutical drugs as well as traditional Chinese drugs may not be suitable for continuous intake or use.

### PRIOR ART DOCUMENTS

### Patent Documents

Patent Document 1: Japanese Unexamined Patent Application Publication No. 8-333244
Patent Document 2: Japanese Unexamined Patent Application Publication No. 2009-13173

### SUMMARY OF THE INVENTION

### Problems to be Solved by the Invention

Therefore, an object of the present invention is to provide a composition which can be applied to the pharyngeal region in a simple and easy manner without causing uneasiness or discomfort, which is effective in preventing or ameliorating snoring and excellent in safety and which can be taken or used continuously.

### Solutions to the Problems

The present inventor has made intensive investigations in order to solve these problems, and found that a composition comprising olive oil, sunflower oil and mentha oil can adequately moisten and soften the mucosa of the pharyngeal region to prevent or ameliorate snoring satisfactorily and can be applied to the pharyngeal region by oral or buccal application in a simple and easy manner without causing uneasiness or discomfort.

The present invention relates to the following embodiments [1] to [18].

[1] A composition for preventing or ameliorating snoring, comprising olive oil, sunflower oil and mentha oil.
[2] The composition according to [1], comprising olive oil in an amount of 2% to 40% by weight, sunflower oil in an amount of 0.5% to 10% by weight and mentha oil in an amount of 0.3% to 5% by weight.
[3] The composition according to [1], wherein the weight ratio of olive oil:sunflower oil:mentha oil is 16:4:1 to 4:1:1.
[4] The composition according to any of [1] to [3], further comprising a vitamin.
[5] The composition according to [4], wherein the vitamin is one or more kinds selected from the group consisting of vitamin B₆, vitamin C and vitamin E.
[6] The composition according to any of [1] to [5], wherein the composition is in liquid form.
[7] The composition according to any of [1] to [5], wherein the composition is in a form suitable for spraying onto the pharynx.
[8] An agent for preventing or ameliorating snoring, comprising the composition according to any of [1] to [5].
[9] The agent according to [8], wherein the agent is an oral liquid formulation.
[10] The agent according to [8], wherein the agent is a pharyngeal spray.
[11] A food for preventing or ameliorating snoring, comprising the composition according to any of [1] to [5].
[12] The food according to [11], wherein the food is in liquid form.
[13] The food according to [11], wherein the food is in spray form.
[14] The food according to any of [11] to [13], wherein the food is a health food, a food for special dietary uses, or a dietary supplement.
[15] A process for the preparation of a composition according to any of [1] to [5], comprising mixing olive oil, sunflower oil and mentha oil, optionally with any other components of the composition, in any order.
[16] The process according to [15], further comprising dissolving, suspending or dispersing the olive oil, sunflower oil and mentha oil in a solvent.
[17] A method of preventing or ameliorating snoring, comprising administering to a patient a composition according to any of to [5].
[18] The method according to [17], wherein the composition is applied directly to the pharyngeal region.

### Effects of the Invention

The composition for preventing or ameliorating snoring of the present invention can be applied to the pharyngeal region in a simple and easy manner without causing uneasiness or discomfort and can prevent or improve snoring effectively. Also, the composition for preventing or ameliorating snoring of the present invention is low-irritating and exceedingly safe, so that it is suitable for continuous intake or use for a long term.

### EMBODIMENTS OF THE INVENTION

A composition for preventing or ameliorating snoring of the present invention (hereinafter, sometimes referred to as a “composition of the present invention”) comprises olive oil, sunflower oil and mentha oil.

Olive oil used as an active ingredient in the composition of the present invention is a vegetable oil obtained from the fruit of the olive (Olea europaea) and contains oleic acid in abundance as a constituent fatty acid of the fat and/or oil. For the purpose of the present invention, olive oil (Oleum Olivae) specified in the Japanese Pharmacopoeia is suitably used.

Sunflower oil is a fat and/or oil obtained from the seed of the sunflower (Helianthus annuus) and contains mainly oleic acid and linoleic acid as constituent fatty acids of the fat and/or oil. For the purpose of the present invention, one having properties suitable as food or the like is preferably used.

Mentha oil is an essential oil obtained by steam distillation of the aerial part of plants in the mint family (Lamiaceae genus), including the Japanese peppermint (Mentha arvensis L. var. piperascens Malinvaud) and peppermint (Mentha piperita) that include menthol as a component. For the purpose of the present invention, mentha oil (Oleum Menthae Japonicae) specified in the Japanese Pharmacopoeia or the like, the menthol content of which is 30% by weight or more, can be preferably used.

In the composition of the present invention, as the above-mentioned olive oil, sunflower oil and mentha oil, it is possible to use fats and/or oils and essential oils obtained by appropriately extracting and collecting the fruit of the olive, the seed of the sunflower, the aerial part of the Japanese peppermint and the like; however, it is convenient to use commercially available products that conform to the above-mentioned standards or the like.

The composition of the present invention preferably comprises olive oil in an amount of 2% to 40% by weight, sunflower oil in an amount of 0.5% to 10% by weight and mentha oil in an amount of 0.3% to 5% by weight, and it is more preferred to contain olive oil in an amount of 4% to 20% by weight, sunflower oil in an amount of 1% to 5% by weight and mentha oil in an amount of 0.6% to 2.5% by weight.

In order to obtain a satisfactory effect of preventing or ameliorating snoring, the composition of the present invention preferably comprises olive oil, sunflower oil and mentha oil in a weight ratio of 16:4:1 to 4:1:1, more preferably a weight ratio of 12:3:1 to 6:1:1.

The total content of olive oil, sunflower oil and mentha oil in the composition of the present invention is preferably 2.8% to 55% by weight, more preferably 9% to 27.5% by weight.

The composition of the present invention preferably further comprises a vitamin, from the viewpoint of imparting a moisturizing property, an anti-inflammatory effect or the like to the mucosa of the pharyngeal region.

As the vitamin, vitamin A, vitamin B group, vitamin C and vitamin E can be used.

Examples of vitamin A include retinol; retinol derivatives such as retinoic acid, retinol acetate and retinol palmitate; and vitamin A oil which is a mixture of them.

Examples of vitamin B group include vitamin B₁ and derivatives thereof such as thiamine, thiamine hydrochloride and thiamine pyrophosphate; vitamin B₂ and derivatives thereof such as riboflavin and riboflavin butyrate; niacin such as nicotinic acid and nicotinamide; pantothenic acid and derivatives thereof such as pantothenic acid, pantothenyl alcohol and pantethine; vitamin B₆ and derivatives thereof such as pyridoxine hydrochloride, pyridoxal, pyridoxal phosphate and pyridoxamine; biotin; folic acid and derivatives thereof such as folic acid, dihydrofolate and folinic acid; and vitamin B₁₂ such as cyanocobalamin, hydroxocobalamin and methylcobalamin.

Examples of vitamin C include ascorbic acid, and derivatives thereof such as dehydroascorbic acid and ascorbyl palmitate.

Examples of vitamin E include natural vitamin E such as d-α-tocopherol, d-β-tocopherol, d-γ-tocopherol, d-δ-tocopherol, d-α-tocopherolacetate and tocotrienol; and synthetic vitamin E such as dl-α-tocopherol and dl-α-tocopherol acetate.

In the present invention, it is possible to select and use one or more kinds from the above-mentioned vitamins; however, for the purpose of the present invention, it is preferred to select and use one or more kinds from the group consisting of vitamin B group, vitamin C and vitamin E, and it is more preferred to select one or more kinds from each group of vitamin B group, vitamin C and vitamin E. In addition, it is still more preferred to use vitamin B₆ as a vitamin selected from vitamin B group.

The content of the above-mentioned vitamin in the composition of the present invention is preferably 0.1% to 5.0% by weight, more preferably 0.5% to 1.0% by weight.

The composition for preventing or ameliorating snoring of the present invention is preferably provided in a form that can be applied to the pharyngeal region directly, such as oral application or buccal application, and it can be provided in forms such as liquid, suspension or dispersion, emulsion, jelly, capsule, solid and aerosol form. The composition for preventing or ameliorating snoring of the present invention may be prepared by mixing olive oil, sunflower oil and mentha oil, in any order and preferably in the amounts and/or weight ratios described above.

The composition in liquid, suspension or dispersion form may be prepared by mixing the above-mentioned olive oil, sunflower oil and mentha oil and preferably adding a vitamin thereto and mixing them; however, it is preferably prepared by dissolving, suspending or dispersing the above-mentioned components in an appropriate solvent.

As the solvent, it is preferred to use a polar solvent such as water; lower alcohols such as ethanol; and polyalcohols such as propanediol, butanediol and glycerol in consideration of a moisturizing effect on the pharyngeal region, ease of handling of the composition and the like. It is more preferred to use water and glycerol from the viewpoint of a moisturizing effect and a low irritation potential on the pharyngeal region.

In order to dissolve, suspend or disperse olive oil, sunflower oil and mentha oil in the polar solvent satisfactorily, it is preferred to use an emulsifier or a suspending or dispersing agent which can be used in oral pharmaceutical preparations or food.

Examples of the emulsifier or the suspending or dispersing agent include polysaccharides such as gum arabic, sodium alginate and guar gum; saponins such as quillaja extract, soybean saponin and tea seed saponin; lecithins such as enzyme-treated lecithin, enzyme-degraded lecithin, vegetable lecithin, fractionated lecithin and egg yolk lecithin; glycerol fatty acid esters such as glycerol monopalmitate, glycerol monostearate and glycerolmonooleate; sucrose fatty acid esters such as sucrose palmitate, sucrose stearate and sucrose oleate; long-chain fatty acid salts such as magnesium stearate; sorbitan fatty acid esters such as sorbitanmonolaurate, sorbitan monopalmitate, sorbitan monostearate and sorbitan monooleate; organic acid monoglycerides such as acetic acid monoglyceride, lactic acid monoglyceride, citric acid monoglyceride and diacetyl tartaric acid monoglyceride; and polyoxyethylene sorbitan fatty acid esters such as polyoxyethylene (20) sorbitan monolaurate, polyoxyethylene (20) sorbitanmonostearate, polyoxyethylene (20) sorbitan tristearate and polyoxyethylene (20) sorbitan monolaurate, and one or more kinds of them can be selected and used.

In the present invention, among these emulsifiers and dispersing agents, glycerol fatty acid esters, sorbitan fatty acid esters and organic acid monoglycerides are preferably used, organic acid monoglycerides are more preferred, and citric acid monoglyceride is particularly preferred among others, from the viewpoint of stability and the like of the composition.

The composition in emulsion form is prepared by emulsifying olive oil, sunflower oil and mentha oil, as well as optional other oil-soluble components if intended to be contained therein and water-soluble components such as water and glycerol, by the use of the above-mentioned emulsifier. At this time, the composition in emulsion form can be formed as an oil-in-water emulsion or a water-in-oil emulsion, depending on the kind, content and the like of the oil-soluble components and the water-soluble components contained in the composition. Here, mentha oil may be added together with a vitamin to the composition after the other components to be contained are emulsified and the resulting composition is cooled.

The composition in jelly form can be prepared by adding a thickening/gelling agent to the above-mentioned composition in liquid, suspension or dispersion form to cause thickening or gelatinization of the composition.

As the thickening/gelling agent, starch sodium octenyl succinate, carrageenan, xanthan gum, gellan gum, gelatin, agar, sodium carboxymethyl cellulose, microcrystalline cellulose, hydroxypropyl cellulose, hydroxypropyl methyl cellulose, methyl cellulose, pullulan, pectin and sodium polyacrylate can be used.

The composition in capsule form can be prepared by filling a hard capsule with the above-mentioned composition in liquid, suspension or dispersion form or composition in emulsion form, or by encapsulating and forming the composition with a soft capsule base.

As the hard capsule, a capsule prepared from gelatin, hydroxypropyl cellulose or the like is used. As the soft capsule base, one formed by adding glycerol or the like to gelatin so as to increase plasticity is used.

The composition in solid form can be prepared by further adding an excipient, a binder, a disintegrant, a lubricant and the like to olive oil, sunflower oil, mentha oil and a vitamin and granulating the mixture, followed by compression-forming. In the present invention, preferred is a composition that dissolves or disintegrates in the oral cavity.

As the excipient, carmellose, carmellose sodium, crystalline cellulose, starch, hydroxypropyl cellulose and the like are used. As the binder, alpha starch, gelatin, shellac, lactose and the like are used. As the disintegrant, crospovidone, low-substituted hydroxypropyl cellulose, croscarmellose sodium, corn starch and the like are used. As the lubricant, talc, magnesium stearate and the like are used.

The composition in aerosol form can be prepared by filling a container with the above-mentioned composition in liquid, suspension or dispersion form together with liquefied gas or compressed gas, or filling a container equipped with a pump dispenser with the composition, so that the composition is in a form to be sprayed.

For the purpose of the present invention, the composition in liquid, suspension or dispersion form is preferably made in a form to be sprayed as a mist.

To the composition for preventing or ameliorating snoring of the present invention, an antioxidant, an antiseptic, a pH regulator, a flavoring agent, a corrigent, a colorant and the like can be added, as long as the features of the present invention are not impaired.

Examples of the antioxidant include erythorbic acid, sodium erythorbate, γ-oryzanol, quercetin, dibutylhydroxytoluene, sage extract, butylhydroxyanisol, gallic acid and rutin.

Examples of the antiseptic include sodium benzoate, disodium ethylenediaminetetraacetate, sorbic acid, potassium sorbate, sodium dehydroacetate, ethyl parahydroxybenzoate, propyl parahydroxybenzoate, butyl parahydroxybenzoate, sodium pyrosulfite and ε-polylysine.

Examples of the pH regulator include hydrochloric acid, sulfuric acid, sodium sulfate, phosphoric acid, potassium monohydrogen phosphate, potassium dihydrogen phosphate, sodium dihydrogen phosphate, trisodium phosphate, citric acid, trisodium citrate, lactic acid, sodium lactate, potassium hydroxide and sodium hydroxide.

Examples of the flavoring agent include acetophenone, isoeugenol, eugenol, d-carvone, 1-carvone, cinnamic acid, citronellol, 1,8-cineole, cinnamaldehyde, vanillin, d-borneol, linalool and limonene.

Examples of the corrigent include sweetening agents such as aspartame, acesulfame potassium, licorice extract, xylitol, sodium saccharin, sucrose, sucralose, stevia extract and d-sorbitol; and bittering agents or acidulants such as adipic acid, caffeine, gentian extract, succinic acid, acetic acid, 1-tartaric acid, 1-sodium tartrate, phytic acid and dl-malic acid.

Examples of the colorant include inorganic pigments such as iron sesquioxide and titanium dioxide; natural colors such as annatto color, turmeric color, α-carotene, gardenia yellow color, chlorophyll, saffron, strawberry color, tamarind color, grape juice and blueberry color; tar colors such as food yellow No. 4, food yellow No. 4 aluminum lake, food blue No. 1, food blue No. 1 aluminum lake, food blue No. 2, food blue No. 2 aluminum lake, food red No. 102, food red No. 104, food red No. 2, food red No. 2 aluminum lake, food green No. 3 and food green No. 3 aluminum lake.

The composition for preventing or ameliorating snoring is taken or used such that the total amount of olive oil, sunflower oil and mentha oil is preferably in an amount of 50 mg to 600 mg, more preferably 100 mg to 300 mg, per adult per day. Here, this amount of the composition for preventing or ameliorating snoring may be taken or used at one time or may be divided to be taken or used at several times.

The composition of the present invention has a mild effect and has active ingredients which are low-irritating olive oil, sunflower oil and mentha oil as well as a vitamin, so that it is low-irritating and exceedingly safe to the mucosa of the oral cavity including the pharyngeal region; therefore, it can be taken or used continuously.

The composition for preventing or ameliorating snoring of the present invention may be made into an agent for preventing or ameliorating snoring by using the composition as it is or by allowing a pharmaceutically acceptable carrier to contain the composition. Examples of the pharmaceutically acceptable carrier include liquid carriers such as water, lower alcohols (e.g. ethanol), polyalcohols (e.g. glycerol), and a mixed solution thereof; and powder carriers such as emulsion, gel and excipient powder.

Accordingly, the agent for preventing or ameliorating snoring of the present invention can be provided as oral liquid formulations such as elixirs, suspending agents, emulsifiers and lemonades; syrups; oral jellies; buccal tablets such as troches and chewing gums; tablets such as buccal disintegrating tablets and chewable tablets; sprays such as buccal sprays and pharyngeal sprays; gargles; and mouthwashes.

The agent for preventing or ameliorating snoring in the above-mentioned dosage forms can be produced by using the composition for preventing or ameliorating snoring of the present invention and adding thereto the above-mentioned additives such as emulsifiers or suspending or dispersing agents, thickening/gelling agents, excipients, binders, disintegrants, lubricants, antioxidants, antiseptics, pH regulators, flavoring agents, corrigents and colorants as needed, by a common production method such as a production method described in each article of [2] Preparation, General Rules for Preparations in the Japanese Pharmacopoeia Sixteenth Edition.

The agent for preventing or ameliorating snoring of the present invention is made preferably as an oral liquid formulation or as a pharyngeal spray that can be applied to the pharyngeal region directly, and more preferably as a pharyngeal spray, in consideration of ease and efficiency when applied to the pharyngeal region and continuation of the moisture effect on the pharyngeal region.

When the agent for preventing or ameliorating snoring of the present invention is made as a pharyngeal spray, this spray can be produced by filling a common spray container with the above-mentioned composition of the present invention in liquid, suspension or dispersion form.

The spray container used in the present invention is not particularly limited as long as it is a container suitable for spray application to the pharyngeal region, but preferably a wide-mouthed container equipped with a pump dispenser having a dip tube is used. In order to regulate the application amount of the agent for preventing or ameliorating snoring of the present invention, it is preferred to use a spray container provided with a metered-dose mechanism capable of regulating the spraying amount at a constant level when the pump dispenser is pressed once. As such a metered-dose mechanism, one which is commonly equipped in spray containers used in the field of otorhinolaryngology, may be used.

When the composition of the present invention is provided as a pharyngeal spray by filling a spray container provided with a metered-dose mechanism with the composition, the spraying amount when the pump is pressed once is preferably 0.1 mL to 4.0 mL, more preferably 0.2 mL to 2.0 mL. Also, the pharyngeal spray of the present invention is used by pressing the pump preferably 1 to several times, more preferably about 3 times per administration for an adult. The number of applications per day is generally about 1 to 6 times, preferably about 1 to 4 times, more preferably about 1 to 3 times, in consideration of the above-mentioned intake amount of the composition of the present invention per adult per day, although it changes depending on the age and the degree of snoring of the user. In addition, the pharyngeal spray of the present invention is more effective to be used at bedtime.

In the present invention, as a filling unit for the formulation component of the spray container, a bottle made of glass or plastic resin is generally used. A light-resistant container with colored glass or the like is preferably used to prevent the formulation component from light deterioration. When a plastic resin is used, one which is not permeable to olive oil, sunflower oil and mentha oil is preferably used. As the pump dispenser, one which is made of plastic resin is commonly used.

As the above-mentioned pump dispenser used in the pharyngeal spray of the present invention and the spray container provided with the metered-dose mechanism, one which is commercially available for sprays in the field of otorhinolaryngology can be used.

The pharyngeal spray of the present invention can be preferably produced by filling the filling unit for the formulation component of the spray container with the above-mentioned composition in liquid form of the present invention, being subjected to a process of sterilization and mounting the pump dispenser thereon.

The composition for preventing or ameliorating snoring can be made into a food for preventing or ameliorating snoring by using the composition as it is or adding a food additive to the composition. The food for preventing or ameliorating snoring of the present invention can be provided in a variety of forms such as liquid, emulsion, semisolid and solid form.

Examples of the food in liquid or emulsion form include soups, sauces, dressings and beverages. Examples of the beverages include fruit beverages such as fruit juice beverages and fruit pulp beverages; vegetable beverages such as tomato juice, carrot juice and vegetable juice; milk; soy milk; lactobacillus beverages; coffee; cocoa beverages; and energy drinks.

Examples of the food in semisolid form include jelly, jam, yogurt and cream.

Examples of the food in solid form include confectionery such as confectionery tablets, e.g. drops, candies, throat drops, chewing gums, soda-pop candies and mint tablets; powdered beverages such as powdered juice, instant coffee and instant cocoa; powdered foods such as instant soup; gummi candies; and encapsulated foods.

In the present invention, it is preferred to provide the food in forms of liquid foods such as beverages; candies such as throat drops; confectionery tablets that disintegrate in the oral cavity; and encapsulated foods, from the viewpoint of easy intake, continuation of the moisture effect on the pharyngeal region and the like. Here, the food in liquid form can be also provided in spray form.

The food for preventing or ameliorating snoring of the present invention can be produced by using the composition for preventing or ameliorating snoring of the present invention and adding thereto additives such as sweeteners, coloring agents, preservatives, thickening stabilizing agents, gelling agents, antioxidants, color formers, bleaches, fungicides, emulsifiers, swelling agents, condiments, acidulants, bittering agents, brighteners, gum bases, nutrient enhancers, agents for production and flavoring agents as needed, by a common food production method. In addition, the food in spray form can be produced according to the above-mentioned production method for the pharyngeal spray.

The food for preventing or ameliorating snoring of the present invention can be provided as health foods such as foods for specified health uses and foods with specific nutritional functions; foods for special dietary uses such as foods for patients and foods for elderly people; and dietary supplements, especially in forms of spray, energy drinks, confectionery tablets, capsules or the like.

The intake amount of the food for preventing or ameliorating snoring of the present invention per adult per day is, on the basis of the total amount of olive oil, sunflower oil and mentha oil, preferably 50 mg to 600 mg, more preferably 100 mg to 300 mg. In the present invention, the intake amount may be taken at one time or may be divided to be taken at several times.

When the food for preventing or ameliorating snoring of the present invention is provided as the above-mentioned health foods, foods for special dietary uses, dietary supplements and the like, the above-mentioned intake amount of the food for preventing or ameliorating snoring of the present invention at one time is preferably contained in the food that is packaged or packed by an intake amount unit per meal. Here, the “food that is packaged or packed by an intake amount unit per meal” means that the amount of food to be taken at one time is packaged or packed in a container such as a bag, box or bottle.

In addition, the composition for preventing or ameliorating snoring of the present invention can be taken by being added to an existing food.

Preferable examples of the existing food include beverages such as tea beverages, fruit juice beverages, coffee and soft drinks; liquid foods such as soup and miso soup; milky foods such as milk and lactic-acid drinks; semisolid foods such as jelly, yogurt and cream; and powdered food such as instant soup, instant miso soup and instant coffee.

The composition for preventing or ameliorating snoring of the present invention is added to the above-mentioned food such that the intake amount per day is within the above-mentioned range.

The present invention will be described in further detail using an example.

### EXAMPLE 1

### Pharyngeal Spray

A liquid composition to prevent or ameliorate snoring was prepared using the formula shown in Table 1. First, a mixture of olive oil, sunflower oil, and monoglyceride citrate was heated to 75° C. to 80° C. Glycerin and purified water were pre-heated and then added gradually, and the resulting mixture was stirred using a homogeniser. After cooling, peppermint oil, vitamin B₆, vitamin C, tocopherol acetate, and a mixture of potassium sorbate and sodium benzoate were added at 40° C. After mixing, the pH was adjusted to between 4.5 to 4.7 using citric acid monohydrate. Spray containers with metered dose spray mechanisms (0.5 mL per spray) were filled with 75 mL each of this composition, sterilisation was performed, and pump dispensers were attached to form pharyngeal sprays.

TABLE 1

| Component | Content (percent by weight) |
|---|---|
| Olive oil | 8.00 |
| Sunflower oil | 2.00 |
| Monoglyceride citrate | 2.20 |
| Glycerin | 12.20 |
| Peppermint oil | 1.00 |
| Vitamin B_{6} | 0.05 |
| Vitamin C | 0.08 |
| Tocopherol acetate | 0.50 |
| Mixture of potassium sorbate | 1.50 |
| and sodium benzoate | |
| Citric acid monohydrate | 0.37 |
| Purified water | 72.10 |
| Total | 100.00 |

### TEST EXAMPLE 1

### Clinical Trial

The following clinical trial was performed using the pharyngeal spray according to the Example 1.

(1) Subjects

Out of a pool of candidates who were healthy but snored in their sleep, twenty subjects (seven women, thirteen male) between the ages of 30 and 67 (with an average age of 47.9) were selected on the basis of the following conditions: (i) does not experience sleep apnoea disorder; (ii) has a partner; and (iii) is not on any medication. Table 2 shows the results of measuring the obesity index (BMI) of the subjects and results from a questionnaire regarding conditions accompanying snoring, the severity of snoring, and dryness of mouth.

TABLE 2

| Obesity (BMI) | |
|---|---|
| Average | 25.2 |
| Standard deviation | 3.5 |
| Range | 17.5-31.2 |
| Number of subjects under 25 | 9 |
| Position during snoring | |
| On back | 2 |
| Different positions | 12 |
| One particular position | 6 |
| Loudness of snoring | |
| Sometimes wakes up partner | 5 |
| Often wakes up partner | 9 |
| Always wakes up partner | 5 |
| No response | 1 |
| Average number of years the | 14 years |
| subject has experienced | |
| snoring during sleep | |
| Dryness of mouth | |
| Sometimes | 8 |
| Often | 7 |
| Always | 3 |
| Never | 2 |

(2) Methodology

The clinical trial was undertaken over the course of two days. The subjects were instructed to use the pharyngeal spray according to the Example 1 (hereinafter referred to as the “test spray”) before bedtime on either the first day or the second day. Whether a subject was to use it on the first day or the second day was determined in a random manner.

The test spray was applied three times (approximately 2 mL) to the pharynx, followed by the subject holding his or her breath for 20 seconds so that the sprayed liquid composition was absorbed in the pharynx. The subjects were prohibited from eating or drinking after use of the test spray.

The subjects then went to bed with their partners. Upon waking up the next morning, the subjects and their partners were asked the questions shown in Table 3. The respondents marked their answers on a Visual Analogue Scale (VAS) ranging from 0 to 100. The subjects' sleep states were recorded on both days using a home sleep testing device (“Embletta Gold” from Embla Systems).

TABLE 3

Questions for subjects
(1) How did you sleep last night? (0: well; 100: badly)
(2) How did you feel when waking up? (0: good; 100: tired)
(3) Was your mouth dry at night or in the morning? (0: not dry;
100: dry)
Questions for partners
(1) Did the subject snore? (0: no snoring; 100: subject snored
continuously)
(2) How loud was the snoring? (0: did not hear snoring; 100:
very loud)

(3) Results

The results of the clinical trial are shown in Table 4. Responses from the subjects and their partners are shown as average scores indicated by subjects and partners. With regard to the subjects' sleep state observation results measured using the home sleep testing device, the number of apnoea and hypopnea events per hour observed during sleep (AHI/h), the average oxygen saturation, and the oxygen desaturation index are shown. These results were evaluated using the Fisher method.

TABLE 4
Category

| | Test | | Difference | | |
|---|---|---|---|---|---|
| | spray | Test | resulting | | |
| | not | spray | from use of | Standard | |
| Category | used | used | test spray | deviation | p-value |
| Subject | | | | | |
| responses | | | | | |
| Sleep state | 58 | 44 | 14 | 16.3 | 0.0010*** |
| Mood in the | 51 | 40 | 11 | 22 | 0.0421* |
| morning | | | | | |
| Dryness of | 46 | 34 | 12 | 25 | 0.0365* |
| mouth | | | | | |
| Partner | | | | | |
| responses | | | | | |
| Snoring | 50 | 40 | 9 | 29 | 0.1821 |
| Loudness of | 49 | 37 | 12 | 24 | 0.0396* |
| snoring | | | | | |
| Subjects' | | | | | |
| sleep state | | | | | |
| AHI/h | 4.7 | 3.4 | 1.2 | 3.2 | 0.1026 |
| Average | 95 | 95.2 | 0.2 | 0.7 | 0.1846 |
| oxygen | | | | | |
| saturation | | | | | |
| Oxygen | 4.7 | 2.7 | 2 | 3.2 | 0.0065** |
| desaturation | | | | | |
| index | | | | | |

*significant to p ≦ 0.05
**significant to p ≦ 0.01
***significant to p ≦ 0.001

In Table 4, it can be seen from the responses from the subjects regarding sleep state that the use of the test spray significantly improves sleep quality (p≦0.001 or p≦0.05). Also, the use of the test spray significantly reduces dryness of mouth felt at night or upon waking (p≦0.05). From the partner responses regarding subject snoring, the use of the test spray exhibited a tendency to reduce the frequency and persistence of the subjects' snoring, although the difference observed was not statistically significant. However, a significant reduction in the loudness of the subjects' snoring resulting from the use of the test spray was observed (p≦0.05).

With regard to the observation results for sleep state, no difference was observed in average oxygen saturation between the use and non-use of the test spray since candidates who did not exhibit sleep apnoea disorder were selected as subjects. While no significant difference was observed, AHI/h tended to decrease slightly with the use of the test spray, and the oxygen desaturation index decreased significantly with the use of the test spray (p≦0.01).

(4) Observations

The results from this clinical trial suggest that the use of the pharyngeal spray according to Example 1 reduces dryness in the oral cavity and consequently prevents or ameliorates snoring and improves sleep quality.

### INDUSTRIAL APPLICABILITY

The present invention provides a composition for preventing or ameliorating snoring that can be applied easily to the pharynx without causing unpleasant sensations or discomfort, that is safe and effective in preventing or ameliorating snoring, and can be taken continuously.

1. A composition for preventing or ameliorating snoring, comprising olive oil, sunflower oil and mentha oil.

2. The composition according to claim 1, comprising olive oil in an amount of 2% to 40% by weight, sunflower oil in an amount of 0.5% to 10% by weight and mentha oil in an amount of 0.3% to 5% by weight.

3. The composition according to claim 1, wherein the weight ratio of olive oil:sunflower oil:mentha oil is 16:4:1 to 4:1:1.

4. The composition according to claim 1, further comprising a vitamin.

5. The composition according to claim 4, wherein the vitamin is one or more kinds selected from the group consisting of vitamin B₆, vitamin C and vitamin E.

6. The composition according to claim 1, wherein the composition is in liquid form.

7. The composition according to claim 1, wherein the composition is in a form suitable for spraying onto the pharynx.

8. An agent for preventing or ameliorating snoring, comprising the composition according to claim 1.

9. The agent according to claim 8, wherein the agent is an oral liquid formulation.

10. The agent according to claim 8, wherein the agent is a pharyngeal spray.

11. A food for preventing or ameliorating snoring, containing the composition according to claim 1.

12. The food according to claim 11, wherein the food is in liquid form.

13. The food according to claim 11, wherein the food is in spray form.

14. The food according to claim 11, wherein the food is a health food, a food for special dietary uses, or a dietary supplement.

15. A process for the preparation of a composition according to claim 1, comprising mixing olive oil, sunflower oil and mentha oil, optionally with any other components of the composition, in any order.

16. The process according to claim 15, further comprising dissolving, suspending or dispersing the olive oil, sunflower oil and mentha oil in a solvent.

17. A method of preventing or ameliorating snoring, comprising administering to a patient a composition according to claim 1.

18. The method according to claim 17, wherein the composition is applied directly to the pharyngeal region.
